(19) United States
(12) Patent Application Publication
(10) Pub. No.: US 2026/0114715 A1
(43) Pub. Date: Apr. 30, 2026

(54) CONNECTOR FOR INSERTING UNIT OF ENDOSCOPE AND FIXING STRUCTURE OF INSERTING UNIT

(71) Applicant: HUNAN VATHIN MEDICAL INSTRUMENT CO., LTD., Xiangtan (CN)

(72) Inventor: Zhenhua ZHOU, Xiangtan (CN)

(73) Assignee: HUNAN VATHIN MEDICAL INSTRUMENT CO., LTD., Xiangtan (CN)

(21) Appl. No.: 19/145,893

(22) PCT Filed: Jan. 12, 2024
(86) PCT No.: PCT/CN2024/071934

§ 371 Date: Jul. 4, 2025

(30) Foreign Application Priority Data
Jan. 31, 2023 (CN) 202310048582.X

(51) Int. Cl. A61B 1/00; A61B 1/018

(52) U.S. Cl. CPC A61B 1/00128; A61B 1/00071; A61B 1/018

(57) ABSTRACT

A connector for an inserting unit of an endoscope and a fixing structure of the inserting unit belong to the technical field of endoscopy. The connector includes a fixing unit and a mounting unit, where an operation channel is provided inside the fixing unit; the operation channel is provided with a first opening located on a surface of the fixing unit; one end of the mounting unit is fixed to the fixing unit, and the other end of the mounting unit is configured to be connected to an inserting unit; the inserting unit is provided with an instrument channel, a traction wire channel, and a wiring channel; and when the mounting unit is connected to the inserting unit, the first opening communicates with the instrument channel, and the traction wire channel and the wiring channel communicate with an environment outside the fixing unit.

### CROSS REFERENCE TO THE RELATED APPLICATIONS

This application is the national phase entry of International Application No. PCT/CN2024/071934, filed on Jan. 12, 2024, which is based upon and claims priority to Chinese Patent Application No. 202310048582.X, filed on Jan. 31, 2023, the entire contents of which are incorporated herein by reference.

### TECHNICAL FIELD

The present disclosure belongs to the technical field of endoscopy, and in particular relates to a connector for an inserting unit of an endoscope and a fixing structure of the inserting unit.

### BACKGROUND

The inserting unit of an endoscope with at least illumination and imaging functions advances into a human body through a natural orifice or a surgical incision to detect the human body's cavity environment. The bending angle of the active bending section located at the front end of the inserting unit of the endoscope is adjusted through a handle located outside the human body, such that the active bending section is deflected in a preset direction to provide a large visual angle for observation.

The inserting unit of the endoscope includes the active bending section at the front end and the passive bending section at the rear end. Existing inserting units include two structural types. In one structural type, the active bending section and the passive bending section are integrally formed. In the other structural type, the active bending section and the passive bending section are separately manufactured. When the integrally formed inserting unit structure is fixed to the handle, the end of the inserting unit is entirely enclosed and secured by a fixing structure to ensure connection stability of the inserting unit. When the instrument channel of the inserting unit is connected to a corresponding device, a short connection tube is provided between the instrument channel and the corresponding device to prevent interference with other channels in the inserting unit, such as the wiring channel and the traction wire channel. The short connection tube can effectively avoid the influence on the wiring channel and the traction wire channel, but in practical applications, to cooperate with the internal space of the endoscope handle, the short connection tube is typically a soft tube, which increases the operational difficulty of the endoscope.

### SUMMARY

To solve the above technical problems, the present disclosure provides a connector for an inserting unit of an endoscope and a fixing structure of the inserting unit. When the inserting unit is fixed, an instrument channel is separated from a wiring channel and a traction wire channel, thereby avoiding the need to dispose a connection tube outside the instrument channel. The present disclosure is implemented as follows:

A connector for an inserting unit of an endoscope includes a fixing unit and a mounting unit, where an operation channel is provided inside the fixing unit, and the operation channel is provided with a first opening located on a surface of the fixing unit; one end of the mounting unit is fixed to the fixing unit, and the other end of the mounting unit is configured to be connected to an inserting unit; the inserting unit is provided with an instrument channel, a traction wire channel, and a wiring channel; and when the mounting unit is connected to the inserting unit, the first opening communicates with the instrument channel, and the traction wire channel and the wiring channel communicate with an environment outside the fixing unit.

In the above technical solution, when the connector fixes the inserting unit, the instrument channel of the inserting unit is separated from other channels, allowing a relevant device to directly enter the instrument channel through the operation channel and the first opening for procedures. This design eliminates the need for a separate connection tube between the operation channel and the instrument channel, reducing the operational difficulty of the endoscope. Additionally, omitting the connection tube improves the responsiveness of operations involving the instrument channel.

Furthermore, the end of the mounting unit connected to the inserting unit is provided with a receiving cavity; the receiving cavity communicates with the first opening; and the receiving cavity is sleeved outside the inserting unit. The design improves fixation stability of the inserting unit.

Furthermore, the receiving cavity is provided with two open ends along an axial direction; a side wall of the receiving cavity is provided with a first gap; and two ends of the first gap communicate with the two open ends, respectively. The presence of the first gap provides a range of variability in the inner size of the receiving cavity, facilitating the mounting of the inserting unit and stabilizing its fixation position.

Furthermore, a length direction of the first gap is parallel to the axial direction of the receiving cavity; alternatively, an arc length of the first gap along a radial direction of the receiving cavity is defined as a width of the first gap; and a central angle corresponding to the width of the first gap is less than 180°. The width of the first gap should not be excessively large to prevent the inserting unit from falling out of the receiving cavity.

Furthermore, the first gap is divided into a constant segment and a diameter-expanding segment along a direction from the receiving cavity to the fixing unit; and a width of the constant segment remains unchanged, and a width of the diameter-expanding segment gradually increases along the direction from the receiving cavity to the fixing unit. After the inserting unit is inserted into the receiving cavity, adhesive fixation is subsequently required. The diameter-expanding segment ensures that the side walls of the receiving cavity on the two sides of the first gap remain sufficiently far from the end of the inserting unit, thereby preventing adhesive overflow from entering the end of the inserting unit and affecting the channel outlet.

Furthermore, an adaptation surface is provided on an outer side wall of the receiving cavity, and the adaptation surface is a planar structure. The adaptation surface is mainly configured to be attached to an inner side wall of an endoscope handle, thereby avoiding displacement of the connector as a whole.

Furthermore, the fixing unit is provided with a connection end surface attached to an end surface of the inserting unit, and the first opening is located on the connection end surface; an edge of the connection end surface is provided with a first curved segment and at least one second curved segment; and the first curved segment is configured to be attached to an edge of the wiring channel, and the second curved segment is configured to be attached to an edge of the traction wire channel. The first curved segment and the second curved segment are mainly configured to prevent height differences arising between the outer side wall of the fixing unit and the channel of the inserting unit, thereby facilitating subsequent wiring.

Furthermore, the operation channel includes a connection segment and an operation segment; One end of the connection segment forms the first opening, and the other end of the connection segment forms a second opening; and the second opening communicates with the operation segment; and a projection surface of the first opening onto a plane where the second opening is located along an axial direction of the connection segment is located within the second opening. The second opening is larger than the first opening to facilitate entry of a relevant device into the instrument channel.

Furthermore, an intersection line between an inner side wall of the connection segment and a plane including an axis of the connection segment is defined as a first intersection line; the first intersection line is structured as a straight line or a curved line; a distance between the first intersection line and the axis of the connection segment along a direction from the first opening to the second opening gradually increases; an intersection line between an outer side wall of the connection segment and the plane including the axis of the connection segment is defined as a second intersection line; the second intersection line is structured as a straight line or a curved line; and a distance between the second intersection line and the axis of the connection segment along the direction from the first opening to the second opening gradually increases. Both the inner side wall and the outer side wall of the connection segment possess inclined guiding features. The inner side wall assists in guiding a relevant device to smoothly move from the second opening to the first opening, and the outer side wall facilitates layout of related wires from the wiring channel and the traction wire channel.

A fixing structure for an inserting unit of an endoscope includes the connector for the inserting unit of the endoscope according to any one of the above paragraphs and the inserting unit, where an active bending section and a passive bending section of the inserting unit are integrally arranged; the receiving cavity is sleeved on an end of the passive bending section far from the active bending section; and the instrument channel communicates with the first opening. When the inserting unit is fixed, the instrument channel of the inserting unit is separated from other channels to facilitate subsequent operations and usage.

The present disclosure has the following beneficial effects:
1. In the present disclosure, when the connector is configured to fix the inserting unit, the instrument channel of the inserting unit is separated from other channels to allow the instrument channel to directly communicate with an operation channel. The wiring channel and the traction wire channel communicate with the environment outside the connector. The present disclosure ensures mutual independence of multiple channels, and eliminates the need for a short connection tube between the operation channel and the inserting unit, reducing operational difficulty during endoscope use.
2. In the present disclosure, the end of the inserting unit is fixed through the receiving cavity to enhance stability at the fixation point. Additionally, the first gap is formed on the side wall of the receiving cavity to facilitate mounting of the inserting unit into the receiving cavity while ensuring connection stability between the receiving cavity and the inserting unit.
3. In the present disclosure, the adaptation surface is provided on the outer side wall of the receiving cavity to assist mounting and positioning, enabling the outer side wall of the receiving cavity to be better attached to the inner side wall of the endoscope handle. The adaptation surface is a planar structure to prevent rotation of the receiving cavity and improve overall stability of the device.
4. In the present disclosure, the first curved segment attached to the edge of the wiring channel and the second curved segment attached to the edge of the traction wire channel are formed at the edge of the connection end surface. The present disclosure eliminates a height difference between the surface of the fixing unit and the edge of the channel, preventing friction between wires extending from the wiring channel and the traction wire channel at channel edges.
5. In the present disclosure, the second opening of the connection segment is larger than the first opening to facilitate entry of instruments into the connection segment. Additionally, both the inner side wall and the outer side wall of the connection segment are inclined. The inner side wall can guide the relevant device to smoothly move from the second opening to the first opening, and the outer side wall assists in smooth laying of the wires extending from the wiring channel and the traction wire channel.

### BRIEF DESCRIPTION OF THE DRAWINGS

To describe the technical solutions in the embodiments of the present disclosure more clearly, the following briefly introduces the drawings required for describing the embodiments or the prior art. Apparently, the drawings in the following description show merely some embodiments of the present disclosure, and persons of ordinary skill in the art may still derive other drawings from these drawings without creative efforts.

FIG. 1 is a first overall structural schematic diagram of a connector according to an embodiment of the present disclosure;

FIG. 2 is a second overall structural schematic diagram of the connector according to an embodiment of the present disclosure;

FIG. 3 is a sectional view of the connector according to an embodiment of the present disclosure;

FIG. 4 is a structural diagram of an inserting unit according to an embodiment of the present disclosure;

FIG. 5 is a schematic diagram of the connector and the inserting unit that match with each other according to an embodiment of the present disclosure;

FIG. 6 is a partial structural detail diagram of the connector and the inserting unit that match with each other according to an embodiment of the present disclosure; and

FIG. 7 is a structural diagram of the connector and the inserting unit that are provided in an endoscope handle according to an embodiment of the present disclosure.

### REFERENCE NUMERALS

100. fixing unit; 110. operation channel; 111. connection segment; 112. operation segment; 121. first opening; 122. second opening; 131. first curved segment; 132. second curved segment; 200. mounting unit; 210. receiving cavity; 211. adaptation surface; 220. first gap; 221. constant segment; 222. diameter-expanding segment; 300. inserting unit; 310. instrument channel; 320. traction wire channel; 330. wiring channel; and 400. endoscope handle.

### DETAILED DESCRIPTION OF THE EMBODIMENTS

The following description provides many different embodiments or examples for implementing different features of the present disclosure. The elements and arrangements described in the following specific examples are only intended to concisely express the present disclosure, and are only for illustration purposes, rather than to limit the present disclosure.

### Embodiment 1

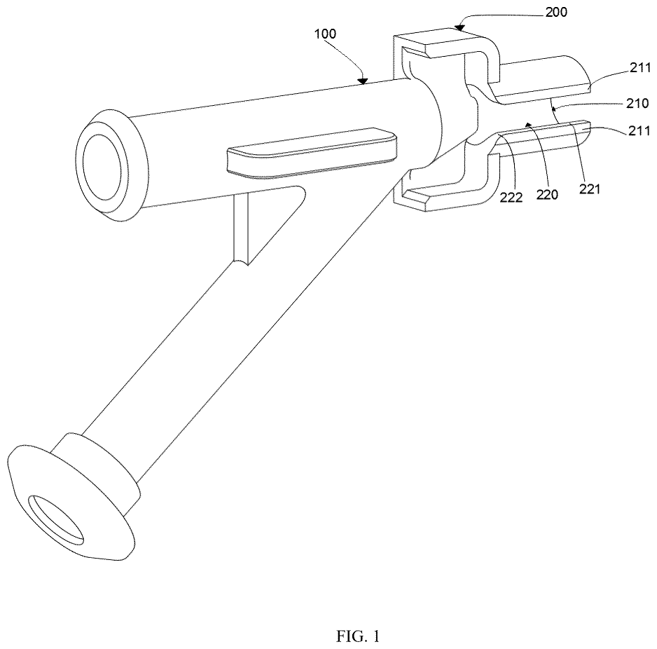

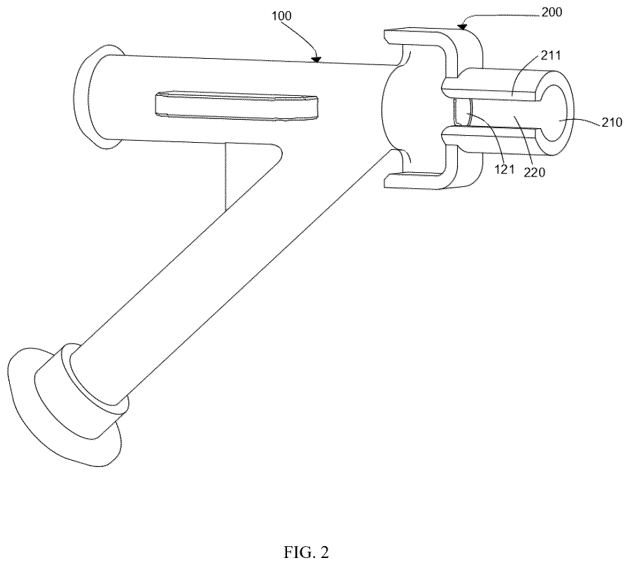

This embodiment provides a connector for an inserting unit of an endoscope. As shown in FIG. 1 to FIG. 7, the connector provided by this embodiment is applicable to integrally formed inserting unit 300 of the endoscope with multiple channels, and specifically includes fixing unit 100 and mounting unit 200. Operation channel 110 is provided inside the fixing unit 100. The operation channel 110 generally has a Y-shaped structure. As shown in FIG. 1 and FIG. 2, the Y-shaped structure includes one end forming first opening 121 located on a surface of the fixing unit 100, one end for use with a negative pressure suction device, and a last end for use with biopsy forceps. One end of the mounting unit 200 is fixed to the fixing unit 100, and the other end of the mounting unit 200 is configured to be connected to the inserting unit 300. The inserting unit 300 is provided with instrument channel 310, traction wire channel 320, and wiring channel 330. When the mounting unit 200 is connected to the inserting unit 300, the first opening 121 communicates with the instrument channel 310, and the traction wire channel 320 and wiring channel 330 communicate with an environment outside the fixing unit 100.

When the connector fixes the inserting unit 300, the instrument channel 310 communicates with the operation channel 110, and the wiring channel 330 and traction wire channel 320 communicate with the external environment, thereby separating the instrument channel 310 from other channels. In prior art, a short connection tube structure is provided between the operation channel 110 and the instrument channel 310. In contrast, the connector structure provided by this embodiment omits the short connection tube, allowing a relevant device to directly enter the instrument channel 310 from the operation channel 110. The design prevents operational difficulty caused by the short connection tube and improves overall usability of the endoscope. Additionally, the omission of the short connection tube enables faster response during operation of the relevant device. Furthermore, when there is a short connection tube provided between the operation channel 110 and the inserting unit 300, the short connection tube will have two connection points that carry risks of leakage and detachment. By omitting the short connection tube, this embodiment avoids risks associated with the connection points and enables a more compact layout within endoscope handle 400.

When the mounting unit 200 is connected to the inserting unit 300, it is sleeved outside the inserting unit 300. The end of the mounting unit 200 connected to the inserting unit 300 is provided with receiving cavity 210. The receiving cavity 210 communicates with the first opening 121. When the receiving cavity 210 is sleeved outside the inserting unit 300, a position of the inserting unit 300 is adjusted to ensure that the instrument channel 310 of the inserting unit 300 communicates with the first opening 121 while the wiring channel 330 and traction wire channel 320 communicate with the external environment.

To improve connection stability between the receiving cavity 210 and the inserting unit 300, an inner diameter of the receiving cavity 210 should not be excessively large. However, if the inner diameter of the receiving cavity 210 is too small, mounting of the inserting unit 300 becomes difficult. To satisfy both requirements of connection stability and ease of assembly, a side wall of the receiving cavity 210 is provided with first gap 220. Two axial ends of the receiving cavity 210 are open ends, with one open end close to the first opening 121 and the other open end far from the first opening 121. The two ends of the first gap 220 communicate with the two open ends, respectively, respectively. The presence of the first gap 220 allows a variable range for an inner size of the receiving cavity 210, facilitating mounting of the inserting unit 300. After side walls on two sides of the first gap 220 are expanded by the inserting unit 300, they exhibit a tendency to close, thereby tightly pressing against an outer surface of the inserting unit 300 and fixing the position of the inserting unit 300.

To facilitate machining, a length direction of the first gap 220 is parallel to an axial direction of the receiving cavity 210. Preferably, an arc length of the first gap 220 along a radial direction of the receiving cavity 210 is defined as a width of the first gap 220. In this embodiment, a central angle corresponding to the width of the first gap 220 is less than 180°. The width of the first gap 220 can vary, but all widths must satisfy the above central angle condition and the width of the first gap 220 should not be excessively large to prevent the inserting unit 300 from falling out of the first gap 220. Generally, the central angle corresponding to the width of the first gap 220 ranges between 60° and 90°.

In this embodiment, the first gap 220 is provided with diameter-expanding segment 222. Along a direction from the receiving cavity 210 to the fixing unit 100, the first gap 220 is sequentially divided into constant segment 221 and the diameter-expanding segment 222. A width of the constant segment 221 remains unchanged, and a width of the diameter-expanding segment 222 gradually increases. Specifically, the width of the diameter-expanding segment 222 gradually increases along the direction from the receiving cavity 210 to the fixing unit 100. When the inserting unit 300 is fixed by the receiving cavity 210, subsequent adhesive bonding is required for reinforcement. To prevent adhesive droplets from falling onto the end of the inserting unit 300, the diameter-expanding segment 222 is provided to ensure side walls of the receiving cavity 210 on both sides of the first gap 220 remain as far away as possible from the end of the inserting unit 300. Specifically, after machining the first gap 220, a chamfering process is directly applied to a position of the first gap 220 close to the end of the inserting unit 300. The first gap 220 formed far from the end of the inserting unit 300 is shown in FIG. 2.

Adaptation surface 211 is provided on an outer side wall of the receiving cavity 210. The adaptation surface 211 is a planar structure. After the connector is provided in the endoscope handle 400, the adaptation surface 211 is attached to an inner side wall of the handle to achieve mounting and positioning. In this embodiment, the adaptation surface 211 can be provided on the two sides of the first gap 220. As shown in FIG. 1 and FIG. 2, the adaptation surface restricts the width of the first gap 220 while providing positioning, thereby preventing changes to the inner diameter of the receiving cavity 210 during use and further enhancing mounting stability of the inserting unit 300.

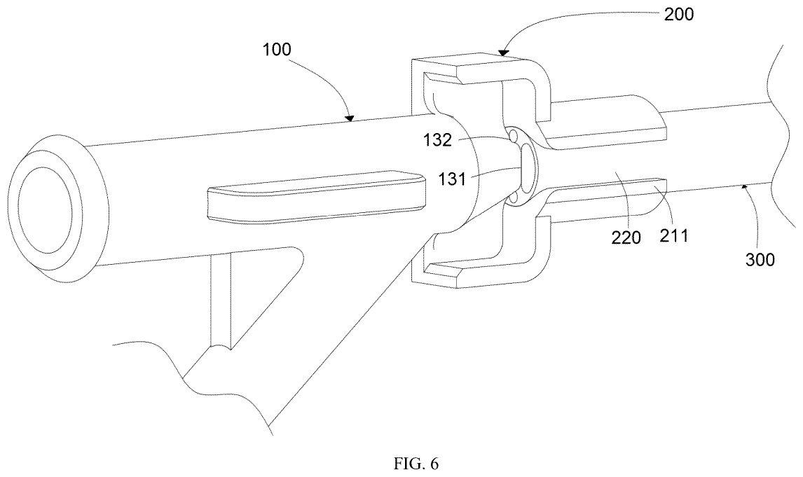

An end surface of the fixing unit 100 attached to an end surface of the inserting unit 300 is a connection end surface. The first opening 121 is located on the connection end surface. An edge of the connection end surface is provided with first curved segment 131 and at least one second curved segment 132. As shown in FIG. 6, the first curved segment 131 is configured to be attached to an edge of the wiring channel 330, and the second curved segment 132 is configured to be attached to an edge of the traction wire channel 320. When the inserting unit 300 is fixed to the fixing unit 100, the first curved segment 131 exhibits no height difference relative to the edge of the wiring channel 330, and the second curved segment 132 exhibits no height difference relative to the edge of the traction wire channel 320. Wires led out from these channels can be directly and normally laid, and will not rub against or be damaged at the edge of the wiring channel 330 during laying.

In this embodiment, the operation channel 110 includes connection segment 111 and operation segment 112. One end of the connection segment 111 forms the first opening 121, and the other end of the connection segment 111 forms second opening 122. The second opening 122 communicates with the operation segment 112. The operation segment 112 includes an end for use with the negative pressure suction device and an end for use with the biopsy forceps. A projection surface of the first opening 121 onto a plane where the second opening 122 is located along an axial direction of the connection segment 111 is located within the second opening 122.

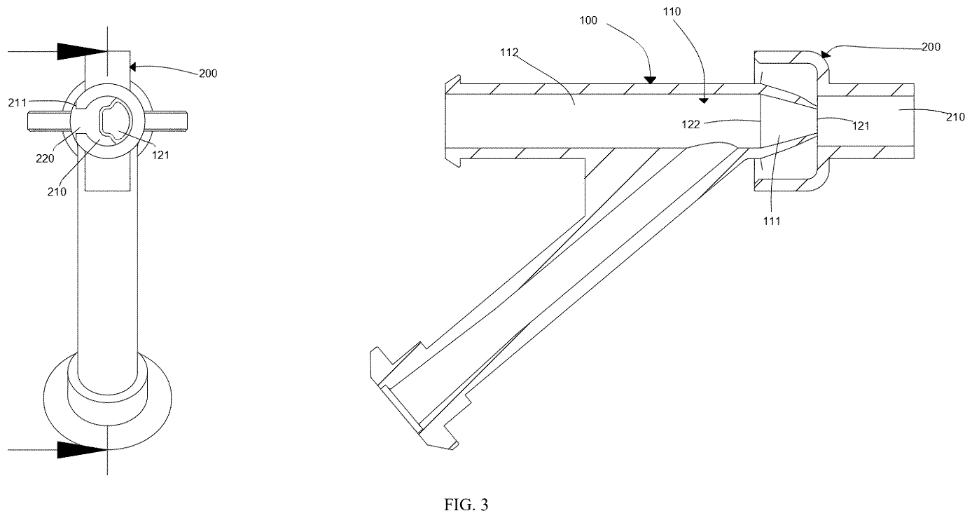

An inner side wall and an outer side wall of the connection segment 111 collectively resemble a side wall structure of a truncated cone, as shown in FIG. 1, FIG. 2, and FIG. 3. However, considering that the first opening 121 must communicate with the instrument channel 310 of the inserting unit 300, to facilitate subsequent use, the shape of the first opening 121 matches a cross-sectional shape of the instrument channel 310. A cross-section of the instrument channel 310 generally exhibits an irregular annular structure. Therefore, a depression or protrusion is formed on the inner side wall of the connection segment 111 close to the first opening 121. Due to the presence of the first curved segment 131 and the second curved segment 132, the outer side wall of the connection segment 111 also includes a depression or protrusion. An intersection line between an inner side wall of the connection segment 111 and a plane including an axis of the connection segment 111 is defined as a first intersection line. The first intersection line is structured as a straight line or a curved line. Along a direction from the first opening 121 to the second opening 122, a distance between the first intersection line and the axis of the connection segment 111 gradually increases. An intersection line between an outer side wall of the connection segment 111 and the plane including the axis of the connection segment 111 is defined as a second intersection line. The second intersection line is structured as a straight line or a curved line. Along the direction from the first opening 121 to the second opening 122, a distance between the second intersection line and the axis of the connection segment 111 gradually increases. Under this configuration, both the inner side wall and the outer side wall of the connection segment 111 possess inclined guiding features. The inner side wall assists in guiding a relevant device to smoothly move from the second opening 122 to the first opening 121, and the outer side wall facilitates layout of related wires from the wiring channel 330 and the traction wire channel 320.

### Embodiment 2

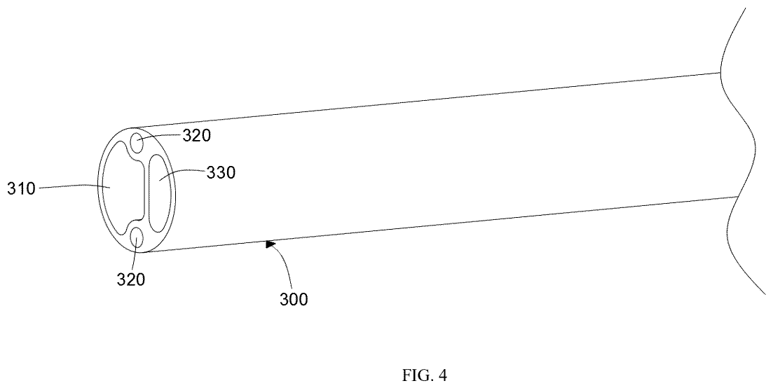

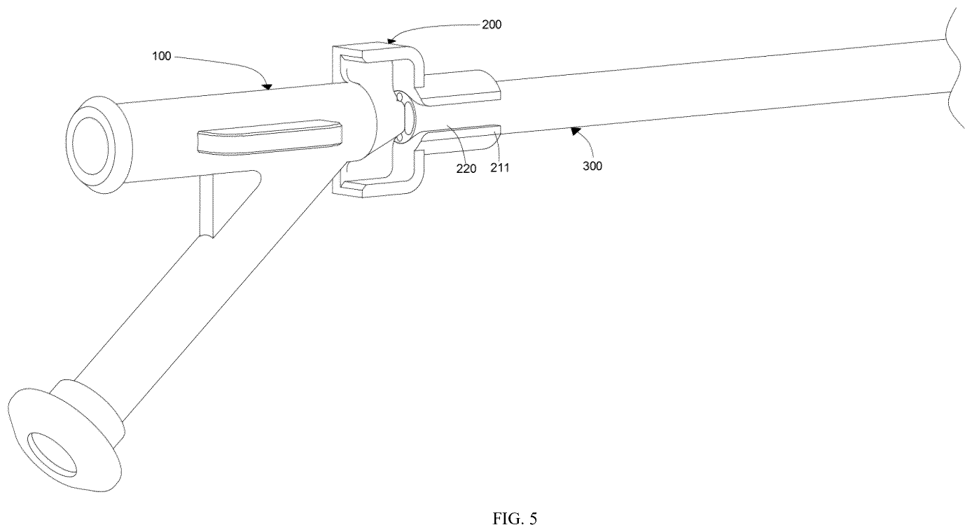

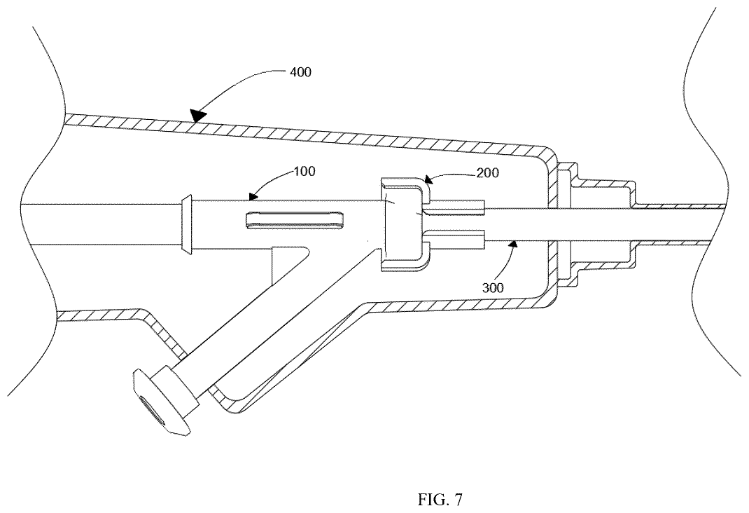

This embodiment provides a fixing structure for an inserting unit of an endoscope. As shown in FIG. 4 to FIG. 7, the fixing structure includes the connector for the inserting unit of the endoscope provided in Embodiment 1 and further includes the inserting unit 300. A structural diagram of the inserting unit 300 is shown in FIG. 4. An active bending section and a passive bending section of the inserting unit 300 are integrally arranged. The receiving cavity 210 is sleeved on an end of the passive bending section far from the active bending section. The instrument channel 310 communicates with the first opening 121, as shown in FIG. 5 and FIG. 6. When the inserting unit 300 is fixed, the instrument channel 310 of the inserting unit 300 is separated from other channels to facilitate subsequent operations and usage. A schematic diagram of the fixing structure provided in the endoscope handle 400 is shown in FIG. 7.

The above are merely preferred embodiments of the present disclosure, and not intended to limit the present disclosure. Any modifications, equivalent replacements, and improvements made within the spirit and principle of the present disclosure should fall within the protection scope of the present disclosure.

What is claimed is:

1. A connector for an inserting unit of an endoscope, comprising:
a fixing unit and a mounting unit, wherein
an operation channel is provided inside the fixing unit, and the operation channel is provided with a first opening located on a surface of the fixing unit;
one end of the mounting unit is fixed to the fixing unit, and the other end of the mounting unit is configured to be connected to an inserting unit; and the inserting unit is provided with an instrument channel, a traction wire channel, and a wiring channel; and
when the mounting unit is connected to the inserting unit, the first opening communicates with the instrument channel, and the traction wire channel and the wiring channel communicate with an environment outside the fixing unit.

2. The connector for the inserting unit of the endoscope according to claim 1, wherein
the end of the mounting unit connected to the inserting unit is provided with a receiving cavity; the receiving cavity communicates with the first opening; and the receiving cavity is sleeved outside the inserting unit.

3. The connector for the inserting unit of the endoscope according to claim 2, wherein
the receiving cavity is provided with two open ends along an axial direction; a side wall of the receiving cavity is provided with a first gap; and two ends of the first gap-communicate with the two open ends, respectively.

4. The connector for the inserting unit of the endoscope according to claim 3, wherein
a length direction of the first gap is parallel to the axial direction of the receiving cavity; alternatively, an arc length of the first gap along a radial direction of the receiving cavity is defined as a width of the first gap; and a central angle corresponding to the width of the first gap is less than 180°.

5. The connector for the inserting unit of the endoscope according to claim 4, wherein
the first gap is divided into a constant segment and a diameter-expanding segment along a direction from the receiving cavity to the fixing unit; a width of the constant segment remains unchanged, and a width of the diameter-expanding segment gradually increases along the direction from the receiving cavity to the fixing unit.

6. The connector for the inserting unit of the endoscope according to claim 2, wherein
an adaptation surface is provided on an outer side wall of the receiving cavity, and the adaptation surface is a planar structure.

7. The connector for the inserting unit of the endoscope according to claim 1, wherein
the fixing unit is provided with a connection end surface attached to an end surface of the inserting unit, and the first opening is located on the connection end surface; and
an edge of the connection end surface is provided with a first curved segment and at least one second curved segment; and the first curved segment is configured to be attached to an edge of the wiring channel, and the second curved segment is configured to be attached to an edge of the traction wire channel.

8. The connector for the inserting unit of the endoscope according to claim 1, wherein
the operation channel comprises a connection segment and an operation segment; one end of the connection segment forms the first opening, and the other end of the connection segment forms a second opening; and the second opening communicates with the operation segment; and
a projection surface of the first opening onto a plane where the second opening is located along an axial direction of the connection segment is located within the second opening.

9. The connector for the inserting unit of the endoscope according to claim 8, wherein
an intersection line between an inner side wall of the connection segment and a plane comprising an axis of the connection segment is defined as a first intersection line; the first intersection line is structured as a straight line or a curved line; and a distance between the first intersection line and the axis of the connection segment along a direction from the first opening to the second opening gradually increases; and
an intersection line between an outer side wall of the connection segment and the plane comprising the axis of the connection segment is defined as a second intersection line; the second intersection line is structured as a straight line or a curved line; and a distance between the second intersection line and the axis of the connection segment along the direction from the first opening to the second opening gradually increases.

10. A fixing structure for an inserting unit of an endoscope, comprising the connector for the inserting unit of the endoscope according to claim 1 and the inserting unit, wherein
an active bending section and a passive bending section of the inserting unit are integrally arranged; the receiving cavity is sleeved on an end of the passive bending section far from the active bending section; and the instrument channel communicates with the first opening.

11. The fixing structure for the inserting unit of the endoscope according to claim 10, wherein in the connector for the inserting unit of the endoscope,
the end of the mounting unit connected to the inserting unit is provided with a receiving cavity; the receiving cavity communicates with the first opening; and the receiving cavity is sleeved outside the inserting unit.

12. The fixing structure for the inserting unit of the endoscope according to claim 11, wherein in the connector for the inserting unit of the endoscope,
the receiving cavity is provided with two open ends along an axial direction; a side wall of the receiving cavity is provided with a first gap; and two ends of the first gap communicate with the two open ends, respectively.

13. The fixing structure for the inserting unit of the endoscope according to claim 12, wherein in the connector for the inserting unit of the endoscope,
a length direction of the first gap is parallel to the axial direction of the receiving cavity; alternatively, an arc length of the first gap along a radial direction of the receiving cavity is defined as a width of the first gap; and a central angle corresponding to the width of the first gap is less than 180°.

14. The fixing structure for the inserting unit of the endoscope according to claim 13, wherein in the connector for the inserting unit of the endoscope,
the first gap is divided into a constant segment and a diameter-expanding segment along a direction from the receiving cavity to the fixing unit; a width of the constant segment remains unchanged, and a width of the diameter-expanding segment gradually increases along the direction from the receiving cavity to the fixing unit.

15. The fixing structure for the inserting unit of the endoscope according to claim 11, wherein in the connector for the inserting unit of the endoscope,
an adaptation surface is provided on an outer side wall of the receiving cavity, and the adaptation surface is a planar structure.

16. The fixing structure for the inserting unit of the endoscope according to claim 10, wherein in the connector for the inserting unit of the endoscope,
the fixing unit is provided with a connection end surface attached to an end surface of the inserting unit, and the first opening is located on the connection end surface; and
an edge of the connection end surface is provided with a first curved segment and at least one second curved segment; and the first curved segment is configured to be attached to an edge of the wiring channel, and the second curved segment is configured to be attached to an edge of the traction wire channel.

17. The fixing structure for the inserting unit of the endoscope according to claim 10, wherein in the connector for the inserting unit of the endoscope,
the operation channel comprises a connection segment and an operation segment; one end of the connection segment forms the first opening, and the other end of the connection segment forms a second opening; and the second opening communicates with the operation segment; and
a projection surface of the first opening onto a plane where the second opening is located along an axial direction of the connection segment is located within the second opening.

18. The fixing structure for the inserting unit of the endoscope according to claim 17, wherein in the connector for the inserting unit of the endoscope,
an intersection line between an inner side wall of the connection segment and a plane comprising an axis of the connection segment is defined as a first intersection line; the first intersection line is structured as a straight line or a curved line; and a distance between the first intersection line and the axis of the connection segment along a direction from the first opening to the second opening gradually increases; and
an intersection line between an outer side wall of the connection segment and the plane comprising the axis of the connection segment is defined as a second intersection line; the second intersection line is structured as a straight line or a curved line; and a distance between the second intersection line and the axis of the connection segment along the direction from the first opening to the second opening gradually increases.
